(19) United States
(12) Patent Application Publication
(10) Pub. No.: US 2012/0136272 A1
(43) Pub. Date: May 31, 2012

(54) Arrangement in a Patient Breathing Tube and a Patient Breathing Tube

(75) Inventors: Anu Kristiina Varis, Vantaa (FI); Anne Tamminen, Vantaa (FI); Janne Ranta, Espoo (FI); Jukka Hakanen, Espoo (FI)

(73) Assignee: GENERAL ELECTRIC COMPANY, Schenectady, NY (US)

(21) Appl. No.: 12/956,622

(22) Filed: Nov. 30, 2010

(51) Int. Cl. A61B 5/097

(52) U.S. Cl. 600/543

(57) ABSTRACT

Arrangement in a patient breathing tube and a patient breathing tube. The breathing tube is provided with a patient end and a ventilator end. The arrangement comprises a sampling conduit having a sampling tip provided at a pre-determined point of the patient end to define a sampling point. The arrangement further comprises a support structure locating the sampling tip of the sampling conduit apart from the inner surface of the patient breathing tube.

### BACKGROUND OF THE INVENTION

The disclosure relates to patient breathing tubes and more closely to sample taking carried out in connection with the use of the patient breathing tube.

During patient care for example in hospitals the amount of tubes, cables and wires used for example in treatment and control steps has caused different problems for decades. Said problems can make work of the doctors and nurse very difficult and can even lead to dangerous situations.

Reducing the amount of tubes, cables and wires going to and from the patient in the OR/ICU would allow more free working space for the doctors and nurses and reduce risk of confusion and unintentional disconnections. Obvious solutions aiming towards this goal are e.g. co-axial tube arrangements or the Limb-o tube where an inner wall creates two separated airflow channels. Logical next step is to insert other related tubes inside the breathing tubes, e.g. the gas sampling line. These inside each other placed tubes may however lead to other problems.

The structures described above are widely used in the field.

As described above the cables, pipes and wires and the structure thereof can cause different problems. As an example of the problems in patient care work problems occurring in interpretation of the information obtained from the gas monitor screen can be mentioned.

In order to correctly interpret the information on the gas monitor screen, the doctor needs to be aware of the gas sampling point's distance from patient. In conventional tubing this is trivial, when the doctor can easily see where the gas sampling tube is connected. If the gas sampling tube and also the gas sampling point point is placed inside the breathing tube, it is may be difficult to estimate gas sampling point's distance from patient. Typically, the closer to the patient the gas sample is taken the better. Also, the gas sampling point should be located so that it is not suctioning water, mucus etc. to the sensor instead of the actual breathing gases.

In some applications, so-called cardiogenic oscillation may cause confusing curves on the patient monitor if the gas sampling point is located too close to the branching unit, for example y-piece branch. Misinterpretation of these variations might lead to mistakes in patient care.

During the anesthesia machine “self-check” the patient end of the breathing tubing is typically connected to a conical stud to block the circuit. The patient connector shall not include any structures that might prevent this.

When the gas sampling line is not connected to a monitor, it must be sealed to prevent the breathing circuit from leaking. Small obstacles entering the sampling tube may affect the monitoring.

### BRIEF DESCRIPTION OF THE INVENTION

The above-mentioned shortcomings, disadvantages and problems are addressed herein which will be understood by reading and understood by reading the following specification.

In one embodiment arrangement in a patient breathing tube having a patient end and a ventilator end, the arrangement comprises a sampling conduit having a sampling tip provided at a pre-determined point of the patient end to define a sampling point. The arrangement further comprises a support structure locating the sampling tip of the sampling conduit apart from the inner surface of the patient breathing tube.

In another embodiment in a patient breathing tube having a patient end and a ventilator end, the patient breathing tube comprises a sampling conduit having a sampling tip provided at a pre-determined point of the patient end to define a sampling point. The patient breathing tube further comprises a support structure locating the sampling tip of the sampling conduit apart from the inner surface of the patient breathing tube.

### BRIEF DESCRIPTION OF THE DRAWINGS

FIG. 1 shows schematically the use of a breathing tube in typical patient care situation,

FIG. 2 shows schematically one embodiment of the arrangement,

FIG. 3 shows schematically another embodiment of the arrangement and

FIG. 4 shows schematically a detail of the breathing tube.

### DETAILED DESCRIPTION OF THE INVENTION

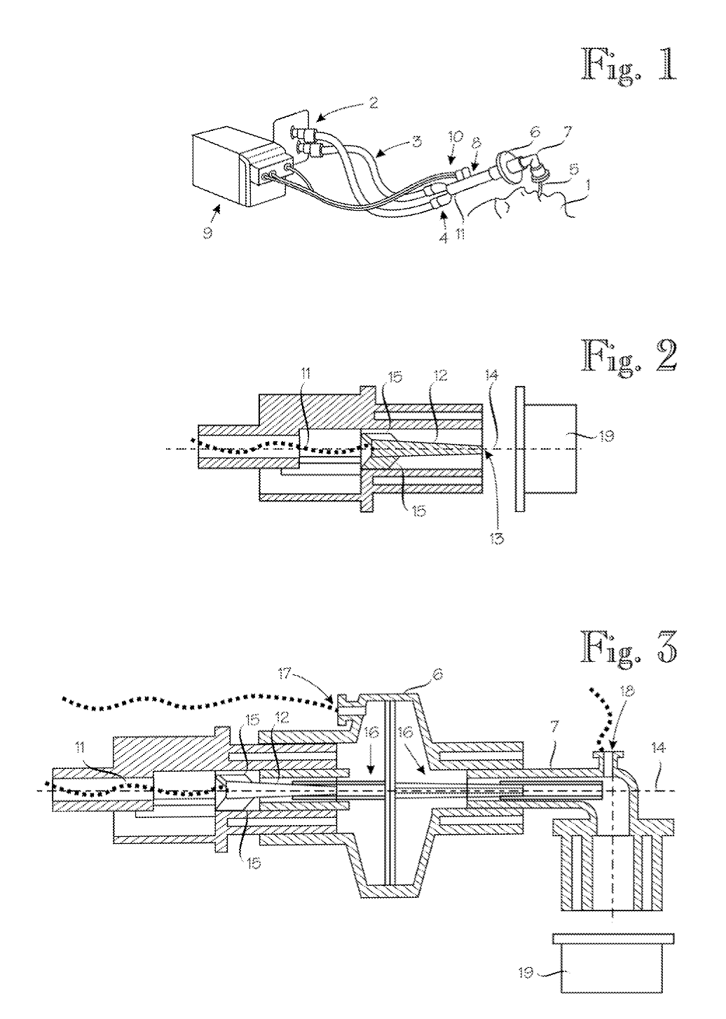

FIG. 1 shows schematically and generally the use of a breathing tube in a typical patient care situation. The breathing tube comprises a patient end and a ventilator end respectively. Reference number 1 shows a patient. Reference number 2 shows generally a ventilator and reference number 3 shows a patient breathing tube. The patient end of the breathing tube is connected to the patient and the ventilator end of the breathing tube is connected to the ventilator.

Reference number 4 shows a branching unit, for example a Y-piece. Reference number 5 shows an intubation tube. Reference number 6 shows a filter and reference number 7 shows an elbow pipe. Reference number 8 shows a flow meter and reference number 9 shows a gas analyzer. Reference number 10 shows flow meter pipes and reference number 11 shows a patient internal gas sampling conduit.

FIG. 1 shows an embodiment using two parallel breathing pipes and a Y-piece as a branching unit. This however not the only possibility. It is naturally possible to use also for example a breathing pipe in which the pipes for inhalation gases and exhalation gases, are arranged coaxially so that one pipe runs inside of the other. When said coaxial type, breathing tube is used the branching unit is designed to match with said coaxial constructions:

The matter described above are known to a person skilled in the art and therefore said matters are not discussed in detail here.

FIG. 2 shows one embodiment of the arrangement. FIG. 2 shows schematically a patient end connector piece of the patient breathing tube. The arrangement shows in FIG. 2 comprises a specific support structure 12 locating the sampling tip 13 apart from the inner surface of the patient breathing tube. The support structure 12 may be for example arranged to locate the sampling tip 13 maximally at a distance of 0.5 of the radius of the patient breathing tube measured from the centerline of the breathing tube, more specifically maximally ⅓ of the radius of the patient breathing tube measured from the centerline of the breathing tube, or even more specifically on the centerline of the patient breathing tube. The centerline of the sampling conduit is shown with a phantom line and reference number 14. In the embodiment of FIG. 2 the sampling tip 13 is placed on the centerline of the patient breathing tube.

In other words the embodiment of FIG. 2 offers a pre-determined measuring point and therefore the personnel using the embodiment has the knowledge of the exact location of the sampling tip 13.

In the embodiment shown the support structure is located into the connector piece of the patient breathing tube. Said embodiment is however not the only possibility but the support structure may also be an integral part of the patient breathing tube for example.

In the embodiment shown in FIG. 2 the sampling conduit 11 runs in the patient breathing tube. The support structure may be made of appropriate fracture-proof material. This is because said support structure must not break into pieces which could be dangerous for the patient if said pieces flow with inspiration gases into the patient's airways for example. As examples of said appropriate materials several plastic materials, for example polypropoylen and polysulfone, may be mentioned.

In the embodiment shown the sampling conduit is a patient internal gas sampling tube. The term sampling conduit must however be interpreted here widely, i.e. said sampling conduit may be pressure conduit or signal wire for example.

As told above in the embodiment shown in FIG. 2 the sampling tip 13 is always fixed in a firm location known to the doctor and away from the bottom of the breathing tube, where it could suction unwanted matter. The support structure 12 is provided with radially extending support parts 15 for supporting the structure in the sampling conduit and preventing the support structure from moving out from the patient breathing tube.

FIG. 2 shows further clearly that the sampling tip 13 is arranged to be in flush with the patient end of the patient breathing tube end. In FIG. 2 the sampling tip is in flush with the patient end connector surface to get as far from the branching unit, for example Y-piece and as close to the patient as possible. In this connection it is important to realize that the sampling tip 13 may not protrude out of the patient end connector without the risk of it blocking e.g. a small size ET-tube.

FIG. 3 shows schematically an another embodiment of the arrangement. In the embodiment shown in FIG. 3 the breathing tube comprises additional components at the patient end. The support structure 12 is provided with extension structure 16. The extension structure 16 extends through the additional components along their centerline. The centerline discussed here is a co-axial with the centerline 14 discussed in connection with the centerline 14 in FIG. 2. Referring to the embodiment of FIG. 3 it must be understood here that in this embodiment the sampling tip 13 may also be placed apart from the inner surface of the patient breathing tube in the same way as described in connection with FIG. 2, i.e. the extension structure 16 need not be placed on the centerline 14 but said extension structure may extend through the additional components so that the sample tip is located away from the inner surface of the patient breathing tube in the way described in connection with the embodiment of FIG. 2.

The additional components discussed above may include for example a filter 6 and an elbow pipe 7.

In case the breathing tubing setup includes additional components, such as filters or elbows, the gas sampling tip can be brought even closer to the patient with special patient end filter and elbow structure. These components have an extension structure for the gas sampling tip 13 fixed on their centerline. These components connected to the coaxial patient tubing with integrated sampling line and tip allow the gas sampling point to be located in the same or better position than in case of conventional separate sampling line. FIG. 3 shows also conventional sampling points. said conventional sampling points have been marked with reference numbers 17 and 18.

Using the elbow part 7 shown in FIG. 3 allows connection to the blocking cone during machine self-check. The coaxial patient tubing with integrated gas sampling tip 13 can also be supplied with a cap 19 mounted on the patient connector that provides necessary blockage during machine self-check and protects the sampling tip 13 from damage and the complete tubing from impurities. Said cap 19 is shown both in FIG. 2 and FIG. 3.

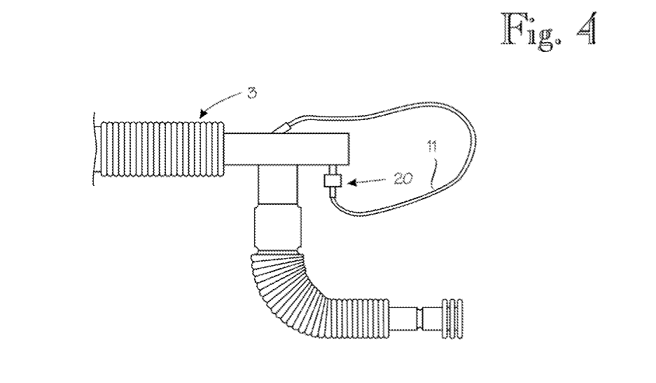

FIG. 4 shows schematically the ventilator end of the patient breathing tube. The ventilator end of the breathing tube may be provided with a parking place 20 for the sampling conduit's machine end connection, i.e. in this case the ventlator end connection. This blocks the sampling conduit and prevents the breathing circuit from leaking, when sampling line is not in use. This allows machine self-check without monitor connection as well as bypassing the whole integrated sampling line if the user decides to use a conventional separate sampling line. Also, when the sampling line is connected to the parking place 20 and protected by the cap 19 in the other end of the breathing tube, impurities cannot enter the tube during transportation and storage.

The written description uses examples to disclose the invention, including the best mode, and also to enable any person skilled in the art to practice the invention, including making using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. Arrangement in a patient breathing tube having a patient end and a ventilator end, the arrangement comprising a sampling conduit having a sampling tip provided at a pre-determined point of the patient end to define a sampling point, the arrangement further comprising a support structure locating the sampling tip of the sampling conduit apart from the inner surface of the patient breathing tube.

2. Arrangement as claimed in claim 1, wherein the support structure is arranged to locate the sampling tip of the sampling conduit maximally at a distance of 0.5 of the radius of the patient breathing tube measured from the centerline of the breathing tube, more specifically maximally ⅓ of the radius of the patient breathing tube measured from the centerline of the breathing tube, or even more specifically on the centerline of the patient breathing tube.

3. Arrangement as claimed in claim 1, wherein the support structure is placed in a patient end connector piece of the patient breathing tube.

4. Arrangement as claimed in claim 1, wherein the support structure is provided with radially extending support parts for supporting the structure in the sampling conduit and preventing the support structure from moving out from the patient breathing tube.

5. Arrangement as claimed in claim 1, wherein the sampling tip is arranged to be in flush with the patient end of the patient breathing tube end.

6. Arrangement as claimed in claim 1, wherein the breathing tube comprises additional components at the patient end, the support structure being provided with extension structure, the extension structure extending through the additional components.

7. Arrangement as claimed in claim 6, wherein the additional components are a filter and an elbow pipe.

8. Arrangement as claimed in claim 1, wherein the sampling conduit is a patient internal gas sampling tube.

9. Arrangement as claimed in claim 1, wherein the support structure is made of fracture-proof plastic material.

10. Patient breathing tube having a patient end and a ventilator end, the patient breathing tube comprising a sampling conduit having a sampling tip provided at a pre-determined point of the patient end to define a sampling point, the patient breathing tube further comprising a support structure locating the sampling tip of the sampling conduit apart from the inner surface of the patient breathing tube.

11. Patient breathing tube as claimed in claim 10, wherein the support structure is arranged to locate the sampling tip of the sampling conduit maximally at a distance of 0.5 of the radius of the patient breathing tube measured from the centerline of the breathing tube, more specifically maximally ⅓ of the radius of the patient breathing tube measured from the centerline of the breathing tube, or even more specifically on the centerline of the patient breathing tube.

12. Patient breathing tube as claimed in claim 10, wherein the support structure is placed in a patient end connector piece of the patient breathing tube.

13. Patient breathing tube as claimed in claim 10, wherein the support structure is provided with radially extending support parts for supporting the structure in the sampling conduit and preventing the support structure from moving out from the patient breathing tube.

14. Patient breathing tube as claimed in claim 10, wherein the sampling tip is arranged to be in flush with the patient end of the patient breathing tube end.

15. Patient breathing tube as claimed in claim 10, wherein the breathing tube comprises additional components at the patient end, the support structure being provided with extension structure, the extension structure extending through the additional components.

16. Patient breathing tube as claimed in claim 15, wherein the additional components are a filter and an elbow pipe.

17. Patient breathing tube as claimed in claim 10, wherein the sampling conduit is a patient internal gas sampling tube.

18. Patient breathing tube as claimed in claim 10, wherein the patient end of the patient breathing tube is provided with a cap for protecting the sampling tip from impurities and damages.

19. Patient breathing tube as claimed in claim 10, wherein the ventilator end of the breathing tube is provided with a parking place for the sampling conduit closing the sampling conduit.

20. Patient breathing tube as claimed in claim 10, wherein the support structure is made of fracture-proof plastic material.
